(19) United States
(12) Patent Application Publication
(10) Pub. No.: US 2024/0207485 A1
(43) Pub. Date: Jun. 27, 2024

(54) Citrate-Based Constructs for Osteochondral Defect Repair

(71) Applicant: Acuitive Technologies, Inc., Allendale, NJ (US)

(72) Inventors: Minh-Tuan Richard Tran, Ho-Ho-Kus, NJ (US); Rui J. Ferreira, Livingston, NJ (US); Michael P. McCarthy, Ho-Ho-Kus, NJ (US)

(73) Assignee: Acuitive Technologies, Inc., Allendale, NJ (US)

(21) Appl. No.: 18/397,950

(22) Filed: Dec. 27, 2023

Related U.S. Application Data
(60) Provisional application No. 63/435,375, filed on Dec. 27, 2022.

(51) Int. Cl. A61L 27/36; A61L 27/12; A61L 27/40; A61L 27/52; A61L 27/56

(52) U.S. Cl. CPC A61L 27/3654; A61L 27/12; A61L 27/40; A61L 27/52; A61L 27/56; A61L 2430/02

(57) ABSTRACT

The present disclosure provides citrate-based constructs for use in the repair of osteochondral defects. The constructs generally include: (i) a citrate component, (ii) a diol component, (iii) a polyol, and (iv) particulate inorganic material. The scaffold may take the form of a 50-90% porous scaffold and may form a polymer network. The scaffold may be soaked in a hyaluronic acid solution and may be freeze-dried to produce a porous construct within the pores of the scaffold. The scaffold may be biphasic, containing a porous section for subchondral bone regeneration and a citrate-based polymer hydrogel for cartilage regeneration. The scaffold may form an implant and the implant may include an inner porous core of a biphasic core-shell construct.

### CROSS-REFERENCE TO RELATED APPLICATIONS

This application claims priority benefit to a U.S. provisional application entitled “Citrate-Based Constructs for Osteochondral Defect Repair,” which was filed on Dec. 27, 2022, and assigned Ser. No. 63/435,375. The entire content of the foregoing U.S. provisional application is incorporated herein by reference.

### BACKGROUND

### 1. Technical Field

The present disclosure is directed to citrate-based constructs for use in the repair of osteochondral defects.

### 2. Background Art

Reported in approximately 20% of all arthroscopic procedures, joint surface lesions (JSLs) involving the articular cartilage and the subchondral bone are clinically very common in orthopedics affecting nearly 600,000 patients annually. JSLs can be superficial, partial-thickness cartilage defects or full-thickness lesions, which do not involve the subchondral bone and cross the osteochondral junction, respectively. JSLs remain a major clinical challenge due to the poor self-healing ability of articular cartilage. If left untreated, JSLs can lead to secondary osteoarthritis (OA). Hence, symptomatic chronic full-thickness defects of the knee joint surface require intervention for symptom relief and to prevent possible evolution towards OA.

An investigation into the natural history and consequence of JSLs in established OA joints recorded chondral lesions in a cohort of patients with osteoarthritis. In this cohort, chondral injuries worsened in 81% of the cases and improved in only 4% over two years [Davies-Tuck, M. L., Wluka, A. E., Wang, Y., Teichtahl, A. J., Jones, G., Ding, C., Cicuttini, F. M, The natural history of cartilage defects in people with knee osteoarthritis, Osteoarthritis and Cartilage, Volume 16, Issue 3, 2007, Pages 337-342]. In a similar prospective study, the presence of cartilage defects in patients with established symptomatic OA was associated with disease severity and was a predictor of joint replacement within four years [Wluka, A. E., Ding, C., Jones, G., Cicuttini, F. M, The clinical correlates of articular cartilage defects in symptomatic knee osteoarthritis: A prospective study, Rheumatology, Volume 44, Issue 10, 2005, Pages 1311-1316].

In summary, JSLs can complicate and accelerate the course of OA. Thus, their treatment may be of functional benefit to the patient, and a need exists for effective treatment modalities.

### SUMMARY

The present disclosure is directed to a synthetic implant/construct designed to treat joint surface lesions. The disclosed biodegradable construct comprises a citrate-based biomaterial that advantageously promotes articular cartilage and subchondral bone regeneration.

Citrate is an inherent molecule in bone anatomy and physiology, playing essential roles in regulating mineral formation and bone metabolism. In biomaterial design, citrate-based polymer functional groups present chemical functional groups for bioceramic interactions, may be reacted according to the present disclosure to prolong release rates, used as conjugation sites for peptide attachment, and as crosslinking sites to create elastomeric properties enhancing tissue regeneration.

Additional features, functions, and benefits of the disclosed scaffolds will be apparent from the following description.

### BRIEF DESCRIPTION OF THE DRAWINGS

To assist those of skill in the art in making and using the subject matter of the present disclosure, reference is made to the appended figures, wherein:

FIG. 1 shows Dubelcco's Modified Eagles Medium (DMEM) extract pH after 72 hours of leaching poly(octamethylene citrate) (POC) containing Bioglass following ISO 10993 standards.

FIG. 2 shows primary chondrocyte proliferation on poly(octamethylene citrate) (POC) scaffolds containing Bioglass compared to tissue culture plate control.

FIG. 3 shows a graphical representation of a porous citrate-based scaffold soaked in hyaluronic acid solution.

FIG. 4 shows a scanning electron microscopy image of porous hyaluronic acid construct within the pores of a citrate-based scaffold after freeze drying.

FIGS. 5 A-C show graphical representations of porous citrate-based scaffolds inserted in the core of a solid citrate based composite with fenestrations between 30-70% to form a core shell construct.

FIGS. 6 A-B show a graphical representation of a porous citrate-based mesh on the chondral side of a core shell construct.

FIGS. 7 A-C show graphical representations of a solid citrate-based composite construct with variable diameter geometry.

FIG. 8 shows primary bovine chondrocyte proliferation on poly(octamethylene xylitol citrate) (POXC) scaffolds containing 60 wt.-% tricalcium phosphate (TCP) and increasing concentrations of Bioglass compared to tissue culture plate control.

FIG. 9 shows a biphasic citrate-based construct containing a porous citrate-based scaffold section for subchondral bone regeneration and a citrate-based hydrogel for cartilage regeneration.

FIG. 10 shows peptides conjugated to the surface of a porous citrate-based scaffold.

FIGS. 11 A-C show graphical representations of a solid citrate-based composite construct with alternate variable diameter geometry.

FIGS. 12 A-C show graphical representations of a solid citrate-based composite construct with alternate variable diameter geometry.

FIGS. 13 A-C show graphical representations of a solid citrate-based composite construct with alternate variable diameter geometry.

FIGS. 14 A-C show graphical representations of a solid citrate-based composite construct with alternate variable diameter geometry.

FIGS. 15 A-C show graphical representations of a solid citrate-based composite construct with alternate variable diameter geometry.

FIGS. 16 A-C show graphical representations of a solid citrate-based composite construct with alternate variable diameter geometry.

FIGS. 17 A-C show graphical representations of a solid citrate-based composite construct with alternate variable diameter geometry.

FIGS. 18 A-C show graphical representations of a solid citrate-based composite construct with alternate variable diameter geometry.

### DESCRIPTION OF EXEMPLARY EMBODIMENTS

The present disclosure provides advantageous citrate-based constructs for use in the repair of osteochondral defects. According to exemplary embodiments, the disclosed construct comprises (i) a citrate component, (ii) a diol component, (iii) a polyol, and (iv) particulate inorganic material. In exemplary embodiments, the citrate component may be selected from the group consisting of citric acid, citrate, and/or an ester of citric acid. In exemplary embodiments, the diol may include butanediol, hexanediol, octanediol, or polyethylene glycerol. In exemplary embodiments, the polyol may include glycerol, beta-glycerol phosphate, and/or xylitol. In forming the disclosed construct, the citrate, diol, and polyol component may form a polymer.

Particulate inorganic material can be added to create composite constructs. In exemplary embodiments, the construct can be fabricated into porous scaffolds to facilitate cell migration, nutrient delivery, and waste removal for tissue regeneration.

The disclosed construct may include particulate inorganic in an amount between 0 and 60 wt. %. In exemplary embodiments, the particulate inorganic material may include one or more of hydroxyapatite, tricalcium phosphate, biphasic calcium phosphate, and Bioglass (BG). BG 45S5 is one bioceramic that can be utilized according to the present disclosure to increase primary chondrocyte cell proliferation, glycosaminoglycan production, and scaffold resorption. BG is composed of 43-47% silica, 22.5-26.5% calcium oxide, 5-7% phosphorus pentoxide, and 22.5-26.5% sodium oxide [Safety Data Sheet—mo-SCI corporation Mo-SCI Corporation. (n.d.). Retrieved May 13, 2022, from mo-sci.com/wp-content/uploads/product-docs/biomaterials/GL0811-SDS.pdf].

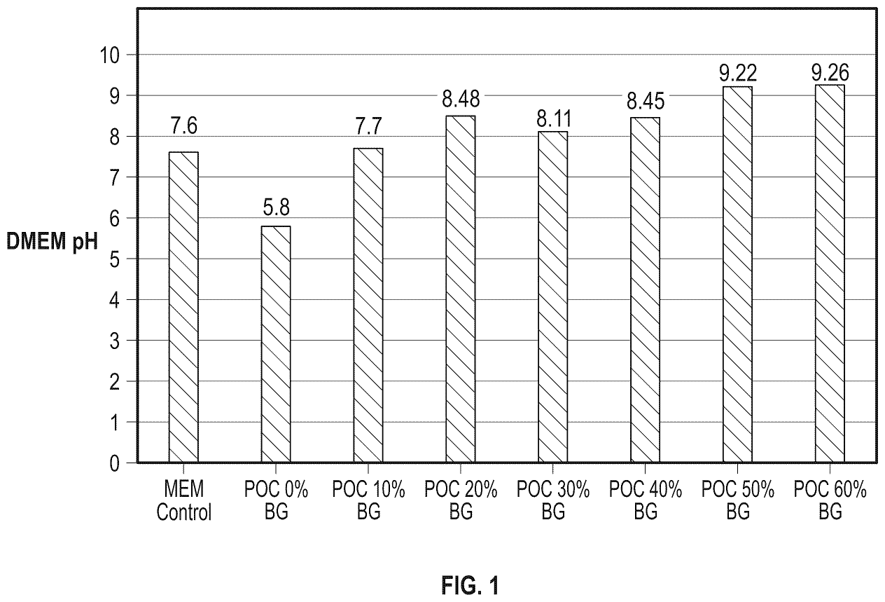

To evaluate the features and benefits of the disclosed construct, citrate-based polymers, including poly(octamethylene citrate) (POC), were combined with 0-40 wt.-% BG and 92 wt.-% sodium chloride to form porous scaffolds. BG can exchange its anions with hydrogen ions in solution, thereby increasing the pH of the surrounding solution to buffer the acidity of POC polymer in solution. As shown in FIG. 1, increased BG concentrations in POC scaffolds increased the alkalinity of cell culture extract media.

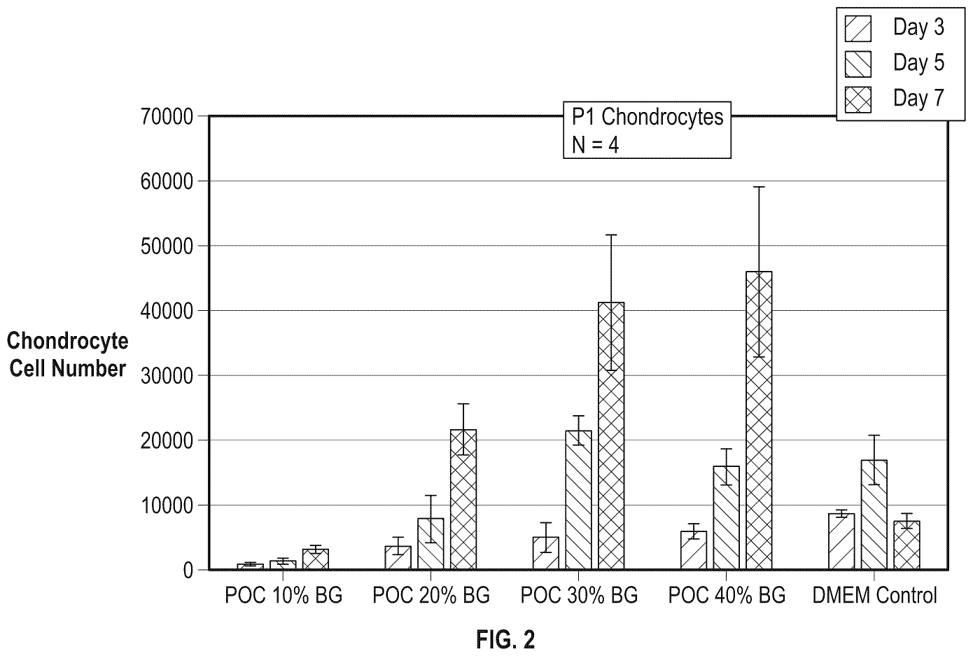

The increase in BG concentration was shown to increase primary chondrocyte proliferation. FIG. 2 shows the proliferation of primary bovine chondrocytes on POC scaffolds composited with 0-40 wt.-% BG over seven days. POC scaffolds containing 20 wt.-% BG and higher allowed for a significantly greater chondrocyte proliferation at 7 days compared to tissue culture plate control.

The bioceramic may also be micro or nano-sized. In exemplary embodiments, the bioceramic may be rod-shaped.

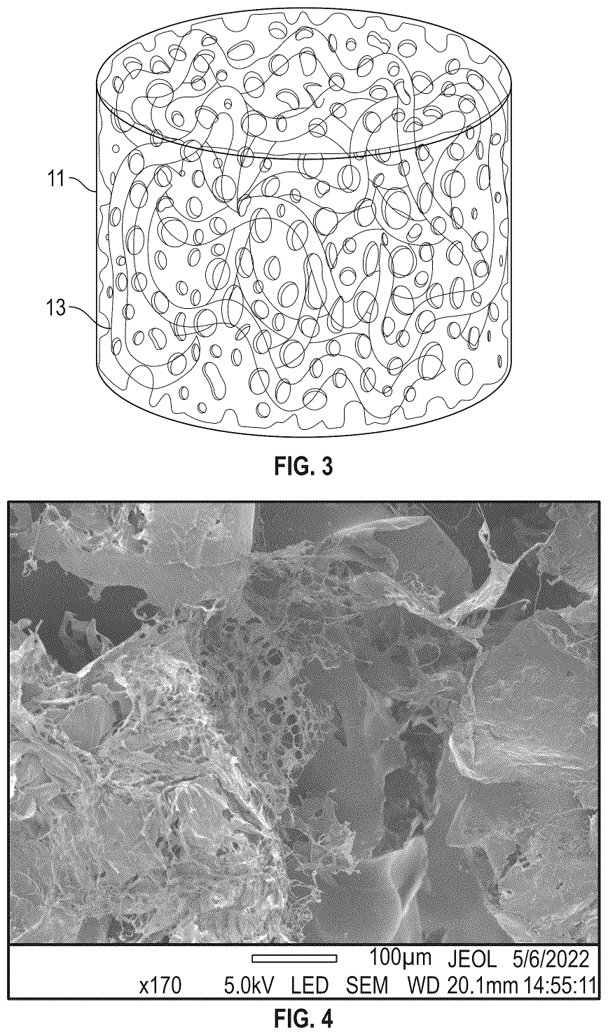

In exemplary embodiments, the scaffold 11 defines a biodegradable scaffold. The scaffold 11 may be soaked in a hyaluronic acid solution 13, e.g., as schematically depicted in FIG. 3.

In exemplary embodiments, the hyaluronic acid-soaked scaffold may be freeze-dried to produce a porous hyaluronic acid construct within the pores of the scaffold, e.g., as shown in the scanning electron microscopy image of FIG. 4.

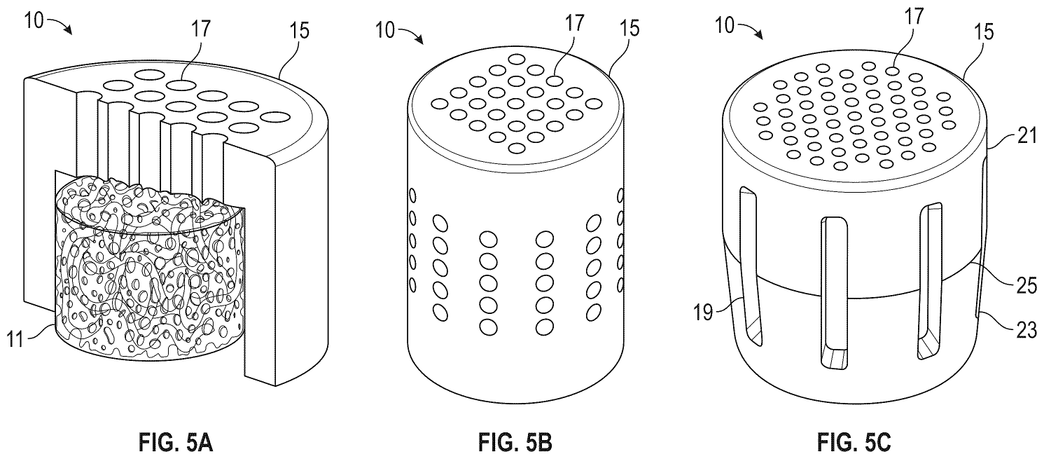

The disclosed construct may advantageously define a porous inner core scaffold 11 of a biphasic core-shell construct 10, e.g., as schematically depicted in FIG. 5 A. As seen in FIGS. 5 A-C, the outer shell 15 can be perforated with circular perforations 17, elongated slots 19, and/or other shaped holes, to allow access to the porous inner core scaffold 11 or provide features to aid in ingrowth of respective cells.

In an exemplary embodiment the outer shell 15 may be open at one end, e.g., as schematically depicted in FIG. 5 A. In a further exemplary embodiment the outer shell 15 may fully enclose the porous inner core scaffold 11, e.g., as schematically depicted in FIGS. 5 B-C, and/or may be made of two or more pieces, e.g., as schematically depicted in FIG. 5 C. The outer shell 15 may including a first part 21 and a second part 23 connected at a seam 25. In an exemplary embodiment, circular perforations 17, elongated slots 19, or other holes may span the seam 25 of the outer shell 15, e.g., as schematically depicted in FIG. 5 C.

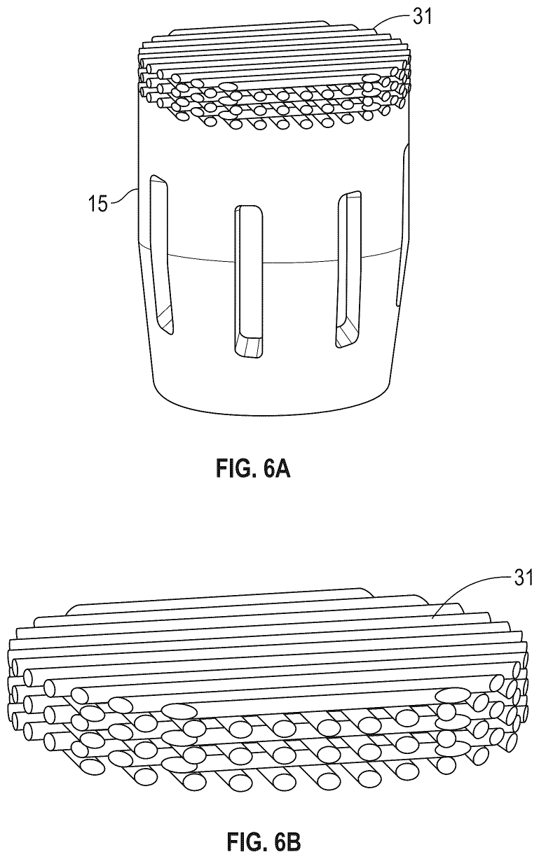

In addition to the configurations described in FIGS. 5 A-C, the disclosed construct may also advantageously define a porous inner core scaffold of a biphasic core-shell construct and a porous mesh 31 on the chondral side of the shell construct, e.g., as schematically depicted in FIG. 6 A. The porous mesh 31 on the chondral side may be fabricated using particulate leaching or 3D printing technologies. It is understood that the porous mesh 31 may be used in addition to any of the shell constructs disclosed herein.

The porous mesh 31 may be made of a plurality of fibers or layers of fibers such that that porous mesh 31 is generally porous, e.g., 50-90% porous. The individual fibers that make up the porous mesh 31 may themselves be porous thereby increasing the porousness of the porous mesh 31 or allowing the fibers to be closer together without reducing the porousness of the porous mesh 31.

In an exemplary embodiment the porous mesh 31 may be used in place of or in addition to circular perforations 17, elongated slots 19, or other holes on the outer shell 15 to promote chondrocyte infiltration and growth factor binding. The porous mesh 31 may be soaked in a hyaluronic acid solution.

In exemplary embodiments the porous mesh 31 may be used independently, e.g., as schematically depicted in FIG. 6 B. The porous mesh 31 may be initially connected to the subchondral bone surface, without having to breach the bone surface, via fibrin glue, sutures, chemical bonding, or another setting or tacky substance, although not limited thereto.

The disclosed construct may take various solid forms, e.g., forms/shapes other than single-diameter cylinders. For example, the disclosed construct 40 may feature regions that define different diameters 41, 43, 45, 47, e.g., constructs wherein the diameter reduces in a direction moving away from the articular surface 49. These subchondral bone penetrating shafts may be fenestrated to allow the integration of new bone growth. These fenestrations may take various forms, e.g., holes and/or slots 51, and may be varied in size, e.g., from 0.5 mm to 2.0 mm.

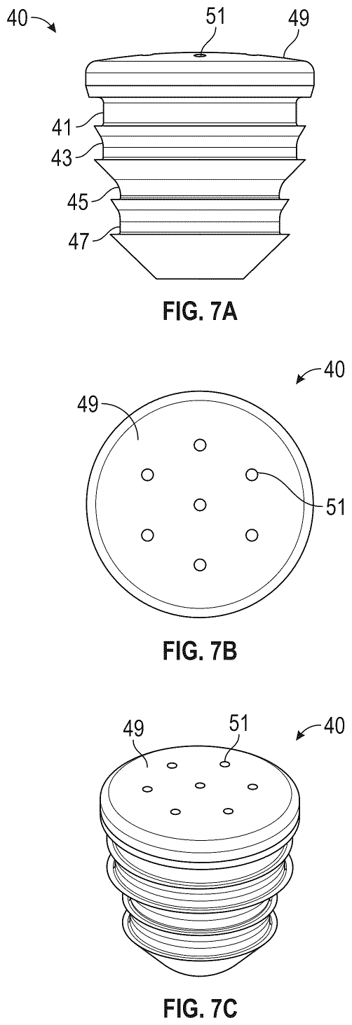

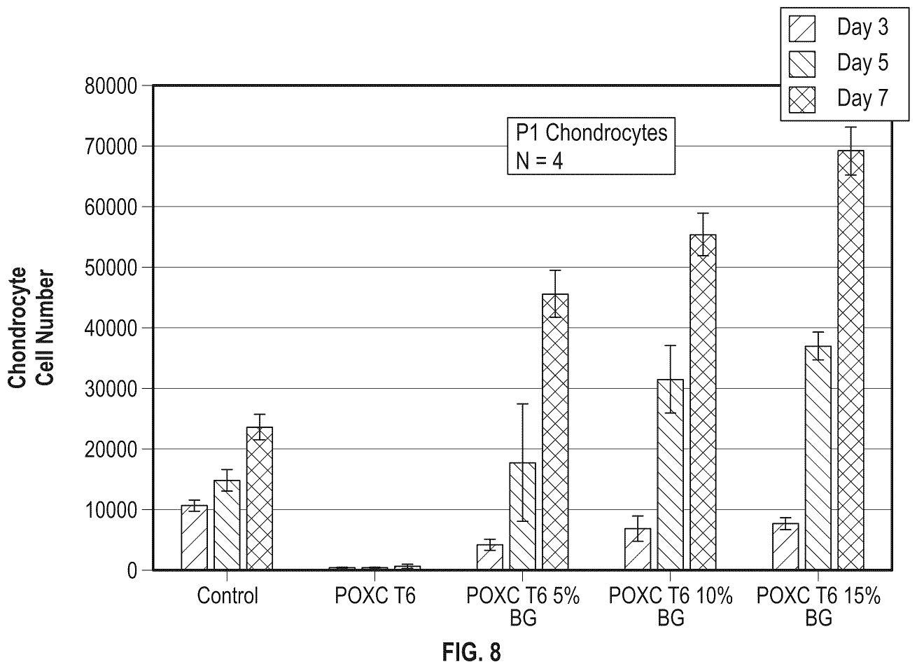

The shell construct, e.g., as shown in FIGS. 5-7, may include a citrate-based composite containing, e.g., 40-65 wt.-% bioceramic, or, e.g., 50-65 wt.-% bioceramic. To evaluate the benefits of this aspect of the disclosed device, citrate-based polymers, including POC with a xylitol addition (POXC), were combined with 60 wt.-% β-tricalcium phosphate (TCP) and 0-15 wt.-% additions of BG. The proliferation of primary bovine chondrocytes was evaluated on these composite formulations. As shown in FIG. 8, the proliferation of these cells increased with increasing amounts of BG.

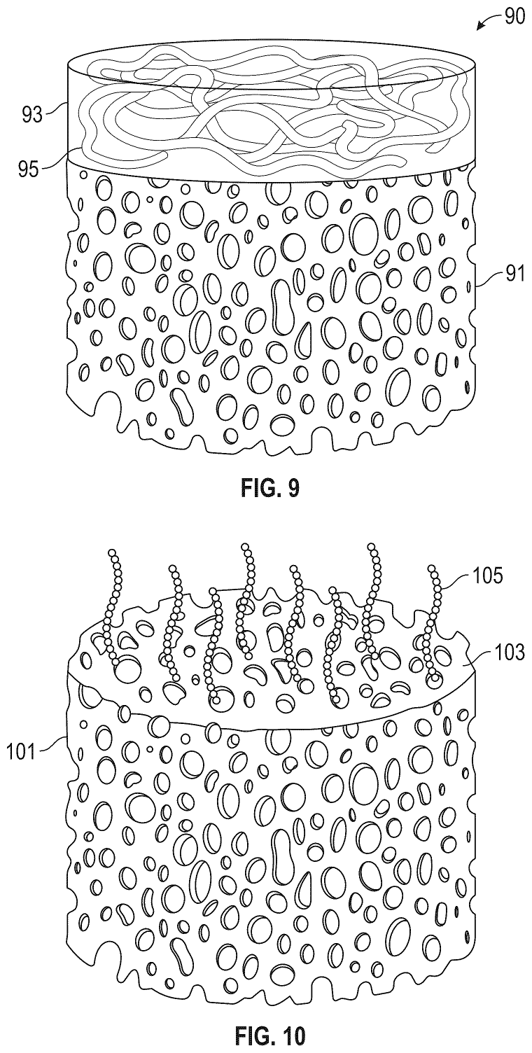

In exemplary embodiments, the disclosed scaffold 90 may be biphasic, containing a porous section 91 for subchondral bone regeneration and a citrate-based hydrogel 93 for cartilage regeneration, e.g., as shown in FIG. 9. In addition, the citrate-based hydrogel 93 may be blended, e.g., with hyaluronic acid 95.

A peptide 105 may be conjugated to the surface 103 of the citrate-based scaffold 101. In exemplary embodiments, a heparin-binding peptide or transforming growth factor-beta mimicking peptides may be conjugated to the surface 103 of the citrate-based scaffold 101, e.g., as schematically depicted in FIG. 10. Growth factor solutions may also be absorbed into the citrate-based scaffold 101.

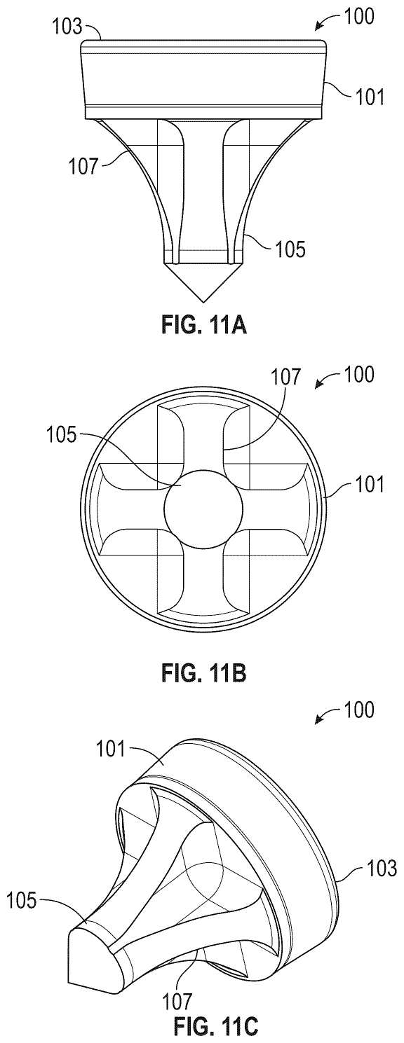

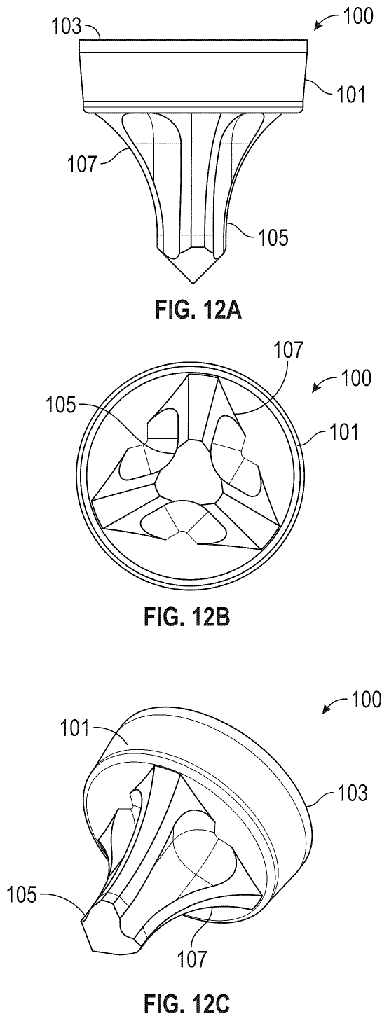

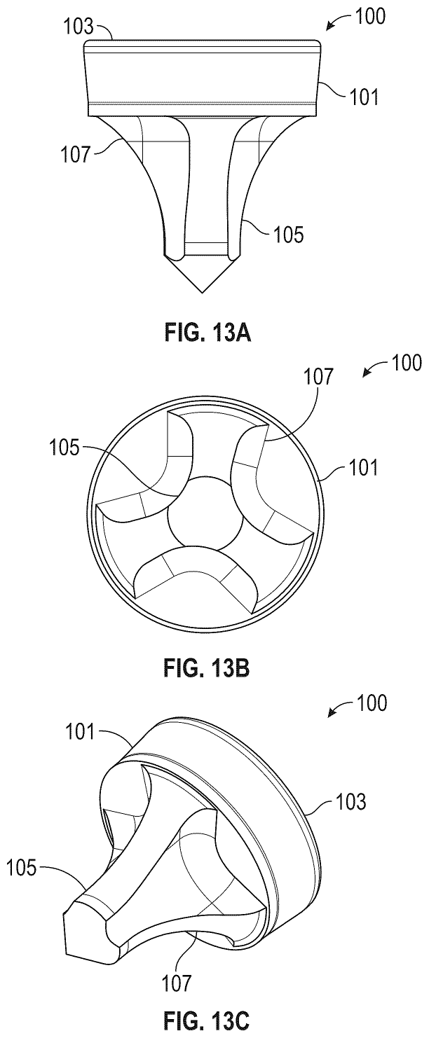

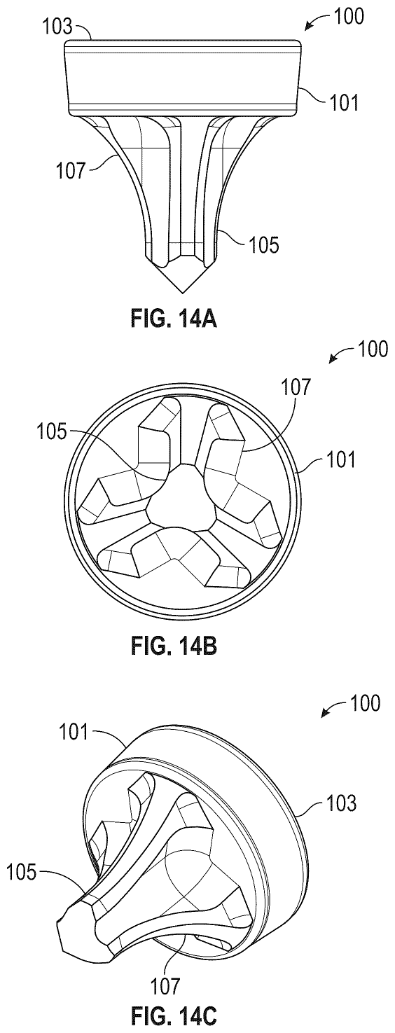

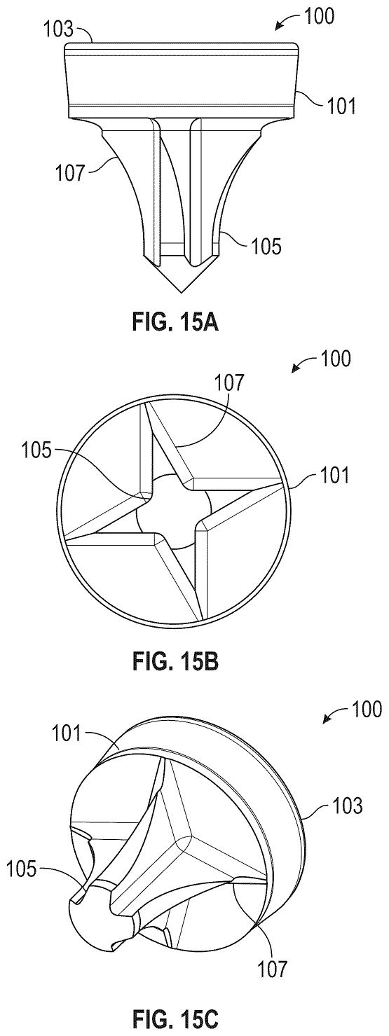

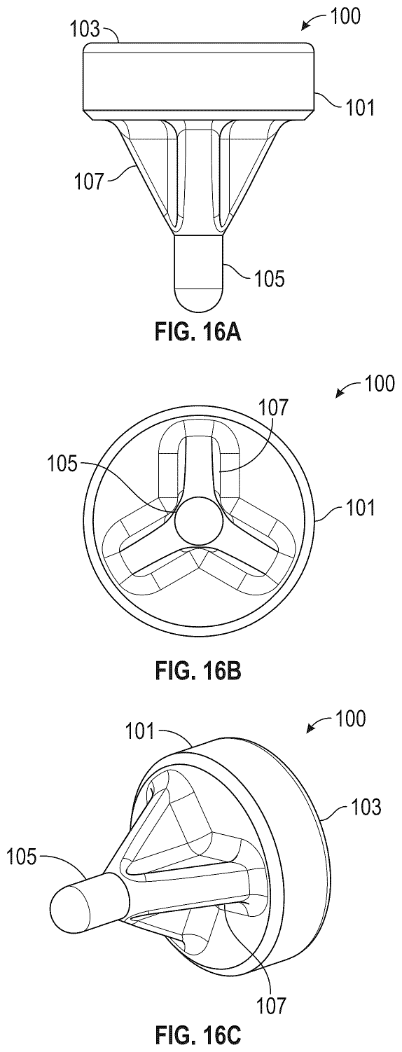

Referring now to FIGS. 11-18 shown are eight exemplary embodiments of a solid citrate-based composite construct with alternative geometries. The citrate-based composite construct 100 may be machined, extruded, mold formed, or printed using 3D printing technologies, although not limited thereto. The composite construct 100 may have a head 101 with a chondral facing side 103. The head 101 may be a single diameter cylinder or may be another shape, e.g., an ellipse, oval, or a truncated cone, although not limited thereto. In exemplary embodiments, the head 101 may be tapered along its axial length such that the chondral facing side 103 is larger than the opposing side of the head 101, e.g., as shown in FIGS. 11-15, and 17-18. This tapered shape may allow for a press seal fit into the user whereby the tapered head 101 is wedged into a cavity in the user's bone. In an embodiment the head 101 may be tapered at an angle between 6 and 10 degrees. In an alternate embodiment the head 101 may be tapered at an angle between 0 and 15 degrees.

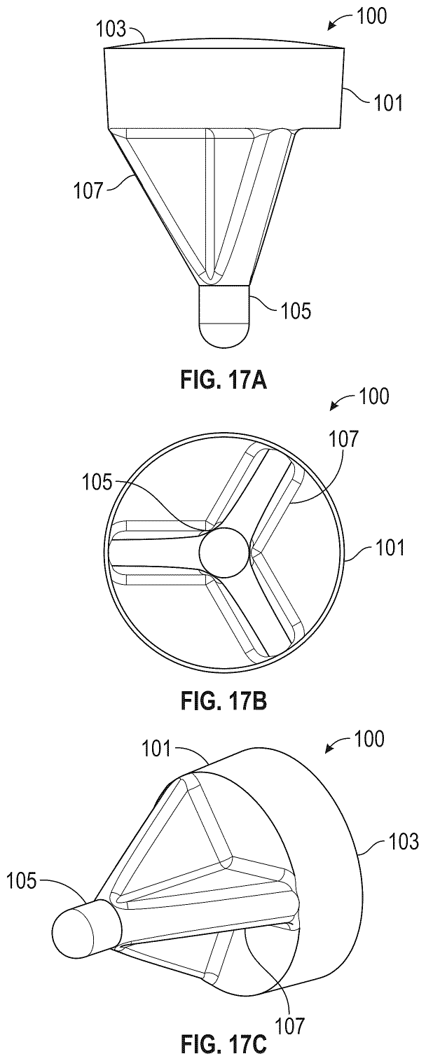

The chondral facing side 103 may be flat, convex, or concave. In an exemplary embodiment, the chondral facing side 103 may be convex matching the surrounding chondral structure, e.g., as shown in FIGS. 17 A-C. In a further exemplary embodiment, the chondral facing side 103 may be flat or concave and may accommodate an additional structure, for example, a porous mesh 31 or a citrate-based hydrogel 93, although not limited thereto.

The composite construct 100 may also have a post or pin 105 extending out of the head 101 opposite the chondral facing side 103. The pin 105 may be supported by a plurality of fins 107. In exemplary embodiments there may be three or four fins 107; however it is appreciated that there may be any number of fins 107, including none, suitable to support the pin 105 and/or provide additional contact surface area of the composite construct 100.

In the exemplary embodiments illustrated in FIGS. 11-18 pins 105 and fins 107 of varying diameter, geometry, number, and orientation are shown. It is understood that any element or configuration of the individual pins 105 and fins 107 illustrated in FIGS. 11-18 may be used alternatively and/or in additional to any other elements or configurations, although not limited thereto, to achieve various desired effects. For example, a pin 105 with a smaller diameter may be desirable as it may require less of a user's bone to be cut away to insert the composite construct 100. Alternatively a pin 105 with a larger diameter may be desirable as it may provide greater structural stability. By way of further example, more or less fins 107 with various geometries may be desirable to support a pin 105 of different diameters and/or provide more/less contact surface area.

Furthermore, the edges and connections of the composite construct 100 may be straight cut, rounded, chamfered, or beveled, although not limited thereto. These edges may provide a better fit of the composite construct 100 in a user or may be used to increase manufacturing efficiency/decrease cost. For example, certain edge finishes on the fins 107 may be more or less prone to chipping depending on the geometry of fin 107 or the radial angle between adjacent fins 107.

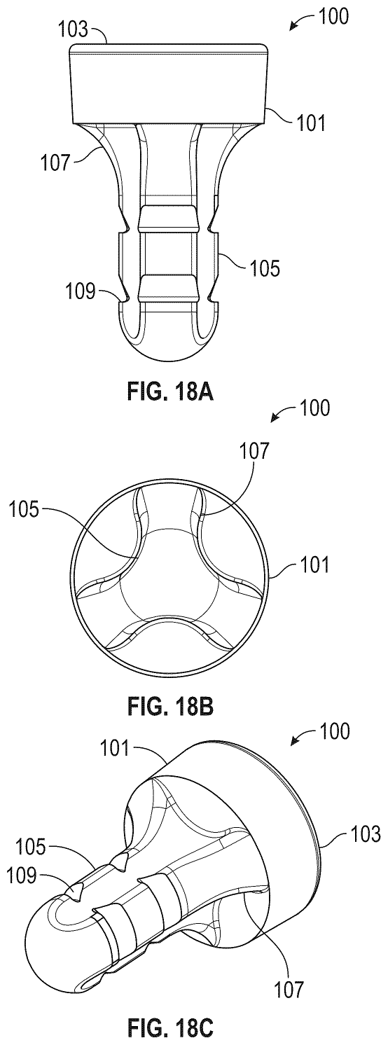

In an exemplary embodiment, the pin 105 or fin 107 may include notches 109, e.g., as shown in FIGS. 18 A-C. The notches 109 may be filled or coated with a growth factor solution to promote ingrowth and adhesion between the composite construct 100 and a user's bone.

An additional consideration is that a composite construct 100, including certain head 101, chondral facing side 103, pin 105, fin 107, notches 109 configurations, may be inefficient or costly to manufacture or may be difficult or impossible to achieve using certain manufacturing processes, i.e., machining versus 3D printing. For example, a pin 105 with a smaller diameter is more prone to breaking during the manufacturing process and adding additional fins 107 decreases the radial angle between adjacent fins 107 making machining more difficult.

While the illustrated embodiments shown in FIGS. 11-18 are solid, it is understood that the composite construct 100 may be hallow and may accommodate a scaffold or other structure similar to those discussed with respect to FIGS. 3, 5, 9, and 10. Additionally, the chondral facing side 103, pin 105, and fin 107 may have circular perforations, elongated slots, and/or other shaped holes, to allow access to an internal structure or provide features to aid in ingrowth of respective cells.

The disclosed scaffolds are generally porous, e.g., 50-90% porous. The scaffolds may contain/define a gradient or biphasic porous structure of two varying pore size ranges. The disclosed scaffold may be conformable and, in exemplary embodiments, may be cut in the operating room.

The disclosed scaffold may swell in liquids, e.g., the disclosed scaffold may swell in liquids by up to 500% to 1500%. The disclosed scaffold generally fully degrades between 6-15 months.

It is appreciated that the various exemplary embodiments, and the components thereof, discussed herein may be used in combination, alternatively, and/or in addition to each other exemplary embodiment, and the components thereof.

Although the present disclosure has been described with reference to exemplary embodiments and implementations, the present disclosure is not limited by or to such exemplary embodiments/implementations.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

While the present disclosure has been described with reference to an exemplary embodiment or embodiments, it will be understood by those skilled in the art that various changes may be made and equivalents may be substituted for elements thereof without departing from the scope of the present disclosure. In addition, many modifications may be made to adapt a particular situation or material to the teachings of the present disclosure without departing from the essential scope thereof. Therefore, it is intended that the present disclosure not be limited to the particular embodiment disclosed as the best mode contemplated for carrying out this present disclosure, but that the present disclosure will include all embodiments falling within the scope of the claims.

1. A construct for use in the repair of osteochondral defects, comprising:
a. a citrate component,
b. a diol component,
c. a polyol, and
d. particulate inorganic material.

2. The construct of claim 1, wherein the citrate component is selected from the group consisting of citric acid, citrate, or an ester of citric acid.

3. The construct of claim 1, wherein the diol comprises butanediol, hexanediol, octanediol, or polyethylene glycerol.

4. The construct of claim 1, wherein the polyol comprises glycerol, beta-glycerol phosphate, or xylitol.

5. The construct of claim 1, wherein the particulate inorganic material comprises one or more of hydroxyapatite, tricalcium phosphate, biphasic calcium phosphate, and Bioglass.

6. The construct of claim 5, wherein the bioceramic is rod-shaped.

7. The construct of claim 1, wherein the citrate, diol, and polyol component form a polymer.

8. A scaffold formed from a construct according to any of the preceding claims.

9. The scaffold of claim 8, wherein the scaffold is a 50-90% porous scaffold.

10. The scaffold of claim 8, wherein the scaffold is a polymer network.

11. The scaffold of claim 8, wherein the scaffold comprises a biodegradable scaffold.

12. The scaffold of claim 8, wherein the scaffold is soaked in a hyaluronic acid solution.

13. The scaffold of claim 8, wherein the scaffold is freeze-dried to produce a porous construct within the pores of the scaffold.

14. The scaffold of claim 8, wherein the bioceramic is present in an amount between 10 and 50 wt.-%.

15. The scaffold of claim 8, wherein the bioceramic is micro or nano-sized.

16. The scaffold of claim 8, wherein a peptide is conjugated to the surface of the citrate-based scaffold.

17. The scaffold of claim 8, wherein a growth factor solution is absorbed onto the citrate-based scaffold.

18. The scaffold of claim 8, wherein the scaffold is biphasic, containing a porous section for subchondral bone regeneration and a citrate-based polymer hydrogel for cartilage regeneration.

19. The scaffold of claim 18, wherein the citrate-based hydrogel is blended with hyaluronic acid.

20. The scaffold of claim 8, wherein a heparin-binding peptide is conjugated to the surface of the citrate-based hydrogel.

21. The scaffold of claim 8, wherein a transforming growth factor beta-mimicking peptide is conjugated to the surface of the citrate-based hydrogel.

22. The scaffold of claim 8, wherein the scaffold contains a gradient porous structure.

23. The scaffold of claim 8, wherein the scaffold is conformable.

24. The scaffold of claim 8, wherein the scaffold can be cut in the operating room.

25. The scaffold of claim 8, wherein the scaffold can swell in liquids 500-1500%.

26. The scaffold of claim 8, wherein the scaffold fully degrades between 6-15 months.

27. An implant formed from a construct according to any of claims 1-7.

28. The implant of claim 27, wherein the implant comprises an inner porous core of a biphasic core-shell construct.

29. The implant of claim 28, wherein the shell construct comprises a citrate-based composite containing 40-65 wt.-% bioceramic or 50-65 wt.-% bioceramic.

30. The implant of claim 27, wherein the implant comprises an inner porous core, a solid outer shell, and a porous component on the chondral side of the implant.

31. The implant of claim 27, wherein the implant comprises a solid component for the subchondral side and a porous component on the chondral side of the implant.
